(19) United States
(12) Patent Application Publication
(10) Pub. No.: US 2003/0233019 A1
(43) Pub. Date: Dec. 18, 2003

(54) Gas to liquid conversion process

(76) Inventor: Steven P. Sherwood, Highlands Ranch, CO (US)

Correspondence Address: Gary J. Connell, SHERIDAN ROSS P.C., Suite 1200, 1560 Broadway, Denver, CO, 80202-5141, US

(21) Appl. No.: 10/391,514

(22) Filed: Mar. 17, 2003

Related U.S. Application Data
(60) Provisional application No. 60/366,068, filed on Mar. 19, 2002.
(60) Provisional application No. 60/410,214, filed on Sep. 11, 2002.

(51) Int. Cl. C07C 2/52; C07C 6/00

(52) U.S. Cl. 585/943; 585/418

(57) ABSTRACT

A process is disclosed for the conversion of lower molecular weight hydrocarbons, such as methane, into higher molecular weight hydrocarbon products, such as hydrocarbons having between 4 and 29 carbons. The process includes forming hydrated electrons, such as by mixing the lower molecular weight hydrocarbons with water and contacting the mixture with an energy source to form hydrated electrons. The hydrated electrons react with the methane to form hydrogen and higher molecular weight hydrocarbon products. Also disclosed is a related process for converting higher molecular weight hydrocarbons to lower molecular weight hydrocarbons by forming a mixture of higher molecular weight hydrocarbons and water and contacting the mixture with an energy source to form hydrated electrons that react with the higher molecular weight hydrocarbons to form hydrogen and lower molecular weight hydrocarbon products.

### CROSS-REFERENCE TO RELATED APPLICATION

This application claims priority under 35 U.S.C. §119(e) from U.S. Provisional Application Serial No. 60/366,068 filed Mar. 19, 2002, entitled “Photolytic Process for the Oxidative Coupling of Methane,” and from U.S. Provisional Application Serial No. 60/410,216 filed Sep. 11, 2002 entitled “Gas to Liquid Conversion Process,” both of which are incorporated herein by reference.

### FIELD OF THE INVENTION

The present invention is a gas to liquid conversion process for the conversion of methane to higher molecular weight hydrocarbons.

### BACKGROUND OF THE INVENTION

Although natural gas has proven to be an excellent fuel for home and industrial heating and for power generation, it is a greatly underutilized resource. Approximately half of the vast natural gas reserves remain untapped throughout the world because the gas is too remote from the market place for cost-effective, conventional pipeline transport. Known reserves of natural gas, of which a principal constituent is methane, rival those of liquid petroleum and are estimated to last for 50 years at the current consumption rate. Estimates of likely, but undiscovered, gas reserves extend the project to over 200 years. When the natural gas thought to lie buried deep beneath the ocean as methane hydrates is added in, the resource could last thousands of years, even with natural gas consumption rates doubling over the next several decades.

An economical process for the conversion of natural gas or methane to an easily transportable liquid product (gas to liquid or GTL) would make it possible to utilize a much larger percentage of the identified reserves.

GTL technology has been a very active research area over the past 50 years, however only two large-scale processes have been demonstrated: 1) the Fischer-Tropsch (FT) process and 2) the methanol-to-gasoline (MTG) process. Both of these processes begin with the costly production of syngas (carbon monoxide and hydrogen) from methane in a reforming operation, which is carried out at a high temperature (typically above 1000° C.) and produces a large amount of excess heat. The reforming process requires large and expensive equipment, making syngas the most capital-intensive process step. For the FT route, the syngas is processed through a second reactor operated to minimize production of methane and ethane and maximize a liquid naphtha product. The FT process also produces water and low temperature heat (less than 230° C.). The MTG route produces a crude gasoline via the intermediate synthesis of methanol.

Alternative GTL processes are presently being developed that do not require the expensive reforming of methane to syngas. Most of these single-step direct-conversion processes involve the oxidative coupling of methyl radicals (CH₃.) at approximately 800° C. and 400 psi. The methyl radicals combine to form light olefins that are oligomerized into gasoline. Increased levels of carbon dioxide in the product gas limit the once-through methane conversion to around 25%.

There remains a need for improved GTL processes that are less expensive and more efficient than known processes.

### SUMMARY OF THE INVENTION

In one embodiment, the present invention includes a process for converting low molecular weight hydrocarbons to higher molecular weight hydrocarbons. The process includes first forming hydrogen and hydroxyl radicals. The process further includes contacting the hydrogen and hydroxyl radicals with a starting material that includes low molecular weight hydrocarbons, whereby the hydrogen and hydroxyl radicals react with the low molecular weight hydrocarbons to form hydrogen and higher molecular weight hydrocarbon products. In one embodiment, the hydrogen and hydroxyl radicals can be formed by contacting hydrated electrons with water. In another embodiment, the low molecular weight hydrocarbons can be selected from methane, ethane, propane, butane and mixtures thereof. Further, the starting material in this process can be natural gas.

In another embodiment of the present invention, a process is provided for converting methane to higher molecular weight hydrocarbons which includes first forming hydrogen and hydroxyl radicals. The hydrogen and hydroxyl radicals are contacted with the methane, whereby a reaction occurs between the methane and hydrogen and hydroxyl radicals to form hydrogen and higher molecular weight hydrocarbon products. In one embodiment, the hydrogen and hydroxyl radicals can be formed by contacting hydrated electrons with water. In this embodiment, the methane can be a gas and the higher molecular weight product can be a liquid. The hydrated electrons can be present in a spur comprising hydrated electrons, H, OH. The higher molecular weight hydrocarbons can include hydrocarbons having between 4 and 29 carbons. Further, the process can be conducted either in the presence or absence of a molecular oxidant. However, when conducted in the presence of a molecular oxidant, the higher molecular weight hydrocarbon products are oxygenated.

In one embodiment, the step of forming hydrated electrons can include contacting a mixture of water and methane with an energy source. In this embodiment, the water can be present as water vapor, which can include the methane. The water can be at a wide variety of temperatures. This mixture of methane and water can be maintained at atmospheric, superatmospheric or subatmospheric pressures. Typically, the methane and water will be present in a mole ratio of between about 1:5 to about 5:1 and preferably in a mole ratio of about 1:1. The energy source can be selected from gamma radiation, ultraviolet radiation, electron beam, and electrical discharge.

In a further embodiment of the present invention, a process is provided for converting high molecular weight hydrocarbons to lower molecular weight hydrocarbons which includes forming hydrogen and hydroxyl radicals and contacting them with high molecular weight hydrocarbons, whereby a reaction occurs in which the high molecular weight hydrocarbons form hydrogen and lower molecular weight hydrocarbon products.

### DETAILED DESCRIPTION OF THE INVENTION

The present invention includes a process of converting lower molecular weight hydrocarbons, such as methane, to higher molecular weight hydrocarbons. The process includes forming hydrogen and hydroxyl radicals and contacting them with lower molecular weight hydrocarbons to form higher molecular weight hydrocarbons. For example, the process can include forming hydrated electrons (e_{aq}⁻), such as by preparing a mixture of lower molecular weight hydrocarbons and water which is then contacted with an energy source. The hydrated electrons react with the lower molecular weight hydrocarbons to form higher molecular weight hydrocarbon products. In the initial stages of process development, a water-catalyzed photochemical process was developed that converts methane into highly branched gasoline-range alkanes (high-octane fuel) and hydrogen. The catalytic properties of water in the photochemical GTL process are believed to be due to the photo-dissociation of water forming hydrated electrons (e_{aq}⁻) and other reactive intermediates. Shortly after photolysis, the e_{aq}⁻ species are localized and solvated as the dipoles of the surrounding water molecules orient around the negative charge of the electrons. The localized electron in rigid matrices (spur) is long lived and is often called a trapped electron or a solvated electron. The dominant reactive species in the spur are e_{aq}⁻, H, OH, all of which can subtract hydrogen atoms from methane and other hydrocarbons to produce organic free radicals that react with other hydrocarbons to form higher molecular weight products.

As referred to herein, the term “hydrocarbons” refers to molecules made up of hydrogen and carbon atoms and can more specifically refer to aliphatic hydrocarbons, either saturated, such as alkanes, and/or unsaturated, such as alkenes and/or alkynes. The term “hydrocarbons” can also specifically refer to cycloaliphatic and/or aromatic hydrocarbons, however, these types of molecules are likely to be present in product streams of the present invention in more minor amounts. The term low molecular weight hydrocarbons typically refers to hydrocarbons having four or fewer carbons, and the term high molecular weight hydrocarbons typically refers to hydrocarbons have five or more carbons.

The starting material for the present invention is preferably a hydrocarbon gas. For example, the gas can be composed of methane, ethane, propane, butane or combinations of ethane, propane, butane and/or methane. Preferably, the gas is natural gas or methane. The methane may be derived from industrial sources as exhaust or recovered from natural deposits although the more highly purified the methane, the greater will be the yield of higher molecular weight products. Importantly, the yield of hydrocarbon products is significantly decreased in the presence of an oxidizing atmosphere. Thus, any oxidizing impurities in the gaseous hydrocarbon starting materials should be carefully avoided, except in the embodiment discussed below in which a controlled amount of a molecular oxidant is used to produce oxygenated hydrocarbons. It is known that the composition of the different natural gases vary depending on the geographic region from which they are isolated. In particular, the nature and concentration of components other than methane as well as the concentration of methane itself in the different types of natural gas are different for various geographic origins. Since the concentration of methane in the different natural gas sources is generally higher than about 75%, the geographic origin of the natural gas and its specific composition are not critical and any natural gas can be used for the present invention.

The process of the present invention includes forming hydrogen and hydroxyl radicals, which function as catalysts for the reactions described herein. For example, the hydrogen and hydroxyl radicals can be formed by contacting hydrated electrons with water. Alternatively, the hydrogen and hydroxyl radicals can be formed by sonication of water. Hydrated electrons can be formed by any currently known process or by any process subsequently developed. Such processes can include radiolysis of water, photolysis of water, high frequency electric discharge, sonolysis of water, and chemical generation of hydrated electrons, such as by the use of Fenton-type reactions. Preferably, hydrated electrons are generated in the presence of water in the vapor state. This is achieved by heating the water either prior to, or after, combining the starting material with the water. The water and the hydrocarbon starting material, particularly in the instance of a methane gas starting material, are initially present in a mole ratio between about 5:1 to about 1:5, more preferably between about 3:1 and about 1:3, and even more preferably, the water and the starting material are initially present in a mole ratio of about 1:1.

After mixing the starting materials, the water can be exposed to an energy source capable of dissociating water to form hydrated electrons. There are many energy sources capable of producing hydrated electrons. Examples include ultraviolet radiation, gamma radiation, electron beam exposure, and electrical discharge, such as corona electrical discharge or dielectric barrier plasma discharge. Exposure of water to these energy sources produces reactive species including e_{aq}⁻, H and OH. These species react to extract hydrogen from the lower molecular weight hydrocarbon molecules present in the starting materials. The resulting hydrocarbon radicals can then react further to remove hydrogen from another hydrocarbon molecule, thereby perpetuating the reaction in a free-radical fashion, or combine with another hydrocarbon radical to form a higher molecular weight hydrocarbon product. If oxygen species are present, the radicals may combine to form oxygen-containing products including esters, hydroxides and carbonyls. For this reason, the reaction is conducted in a neutral or reducing atmosphere while an oxidizing atmosphere is to be avoided, unless there is a desire to produce oxygenated hydrocarbons.

When ultraviolet radiation is used as the energy source, the photocatalytic reactions proceed when the reaction systems are irradiated with ultraviolet-light in wavelength regions shorter than about 380 nm. Preferably, the ultraviolet radiation is provided at a wavelength of about 150 nm to about 280 nm. When photocatalytic production of hydrated electrons is employed, the use of standard photocatalysts can increase the rate of hydrated electron production.

When gamma radiation is used to produce the hydrated electrons, a dose rate of about 10 kRad/min to about 50 kRad/min for about 10 to about 20 hours is sufficient to generate hydrated electrons leading to higher molecular weight hydrocarbon products. Preferably, the dose rate is about 20 kRad/min for about 15 hours.

When an electron beam is used to produce the hydrated electrons, multiple short exposures of an at least about 300 kv electron-beam radiation are sufficient to generate hydrated electrons leading to higher molecular weight hydrocarbon products.

When electrical discharge, (such as corona electrical discharge or dielectric barrier discharge (DBD)) is used to produce the hydrated electrons, an average discharge energy of about 5 kv is sufficient to generate hydrated electrons leading to higher molecular weight hydrocarbon products-.

The reactions of the present invention can be carried out under conditions of controlled pressure. While the reaction may be conducted under subatmospheric, atmospheric or superatmospheric conditions, increasing the pressure above atmospheric conditions increases the yield of higher molecular weight hydrocarbons in the final products. The pressure may be increased to greater than about 50 psi, greater than about 80 psi, or greater than about 10 psi. As noted above however, the reaction proceeds to greater yield if the water is maintained in a vapor state. Therefore, the pressure should be controlled in conjunction with the temperature of the reaction to increase the pressure without driving the water into the liquid phase.

The temperature of the reaction is maintained at greater than about 15° C. although higher temperatures are generally preferred. Temperatures in the range of about 25° C. to about 200° C. are useful with temperatures between about 90° C. and about 110° C. being preferred. Alternatively, the water can be at a temperature in the range of between about 15° C. and about 50° C., or between about 50° C. and about 150° C., or greater than about 150° C. The increased temperature of the reaction increases the production of the higher molecular weight hydrocarbon products by increasing the reaction rate and by helping to maintain the water in the vapor phase. The increase in reaction temperature is limited only by the breakdown of the higher molecular weight hydrocarbon products. For example, methane brought to a temperature above 1,200° C. will break down through a sequence of reactions of dehydrogenations and cyclizations into a mixture of undesirable polyaromatic substances leading to carbon black. However, in the embodiment of producing lower molecular weight products from high molecular weight starting materials, operation at higher temperature ranges, such as in the range of about 50° C. and about 250° C., or greater than about 250° C. can be preferable to ensure vaporization of the higher molecular weight starting materials.

The desired higher molecular weight hydrocarbon products may contain between 4 and 29 carbons. More often however, the products are hydrocarbons having between 9 and 14 carbons. The length of the hydrocarbon chain or the degree of branching may be controlled to some degree by the pressure and temperature of the reaction. As noted earlier however, higher molecular weight products may be broken down in reactions conducted at high temperatures.

Interestingly, the higher molecular weight products of the reaction generally fall within a range of 4 to 20 carbon atoms. Without intending to be bound by any theory, it is believed that this range of carbon atoms within the backbone of the hydrocarbon products results from a buildup of higher molecular weight products while much higher molecular weight products having 20 to 30 or more carbon atoms break down under the reaction conditions. Thus, by starting with higher molecular weight products having in excess of 20 to 30 carbons, the reaction can be conducted to form lower molecular weight hydrocarbon products having carbon atoms within the range of 4 to 20 carbons. All of the reaction conditions and parameters discussed above with regard to the process for forming higher molecular weight hydrocarbons are applicable to this process of forming lower molecular weight hydrocarbons.

The production of lower molecular weight products can be used to refine or recycle high molecular weight hydrocarbon stocks into useful lower molecular weight hydrocarbon fuel sources. For example, spent motor oil, tar, asphalt or refinery discharges may be converted or recycled into useful fuels using this method. Although the purpose of the reaction and the starting materials are different, the reaction conditions are largely unchanged in this process.

The following Examples are provided to illustrate embodiments of the present invention and are not intended to limit the scope of the invention.

### EXAMPLES

### Example 1

### Photochemical Studies

This example demonstrates the process of the present invention in which the energy source used to generate hydrated electrons is ultra violet radiation.

The use of ultra violet radiation in the process of the present invention is described in detail in U.S. Provisional Application Serial No. 60/366,068 filed Mar. 19, 2002, which is incorporated herein in its entirety by this reference.

Continuous-flow photolysis studies were conducted in a 5.7-liter, 4-inch-diameter, 28-inch-long cylindrical stainless steel reactor. The apparatus was designed to accommodate a 32-inch long low-pressure mercury vapor lamp. The reactor was fitted with a 100-Watt Sunlight Systems UV lamp with GE-214 quartz envelope to provide 85% transmittance of 185-nm photon emission.

For the initial test, the reactor was charged with approximately 24 psig ultra-pure methane (99.7%) and heated to 84° C. Upon reaching the target temperature, the ultraviolet light source was energized and methane control valve opened to achieve a methane flow rate of 36 mls/min. Samples of process gas were collected through a septum in the reactor exhaust line and analyzed for hydrogen and light hydrocarbon content (H2, ethane, ethylene, acetylene, CO2 and CO) with a dual-channel MicroGC gas chromatography (GC) using Molecular Sieve 5A PLOT and PoraPLOT U columns. Higher molecular weight products were characterized by a chromatograph/mass spectrometer (GC/MS) with a Restek Rtx-1 column.

In the second study, the reactor was charged with approximately 250 mls deoxygenated deionized water and flushed with methane. The system was heated to 84° C. and additional methane added to a system pressure of 24 psig. Ultra-pure methane was processed through the photoreactor at a flow rate of 34 ml/min. The results from the photochemical studies are summarized in the following table.

TABLE 1
Distribution of Hydrocarbon Products from Photochemical Studies

| | 99.7% Methane | |
|---|---|---|
| Constituent | No Water | Methane and Water |
| GC Results, mole % | | |
| Hydrogen | 0.219 | 3.44 |
| Ethane | 0.035 | 0.17 |
| Carbon Monoxide | Not Detected | Not Detected |
| Carbon Dioxide | Not Detected | Not Detected |
| GC/MS Results, Peak Area % | | |
| Propane | 7.34 | 6.34 |
| 2-Methylpropane | 5.28 | 4.51 |
| 2,2-Dimethylpropane | 12.2 | 15.7 |
| 2-Methylbutane | 2.10 | 4.01 |
| 2,2-Dimethylbutane | 7.04 | 6.41 |
| 2-Methylpentane | 0.97 | 0.40 |
| 2,2-Dimethylpentane | 2.35 | 1.20 |
| 2,2,3-Trimethylbutane | 3.05 | 3.52 |
| 3,3-Dimethylpentane | 2.26 | 1.89 |
| 2,2-Dimethylhexane | 1.83 | 1.45 |
| 2,2,3,3-Tetramethylbutane | 7.61 | 9.67 |
| 2,4-Dimethylhexane | 1.82 | 1.45 |
| 3-Methyl, 3-Ethylpentane | 1.75 | 1.72 |
| 2,2,3,3-Tetramethylpentane | 6.46 | 9.66 |
| 2,2,3,3-Tetramethylhexane | 1.56 | 2.18 |
| 3,3-Diethylpentane | 9.61 | 5.82 |

No unsaturated hydrocarbons, oxygenated hydrocarbons, or aromatic compounds were identified in the reaction products.

### Example 2

### Electron Beam Gas-To-Liquid Studies

The purpose of this electron beam test was to demonstrate that the e_{aq}⁻ is indeed the initiator or catalyst for the chemistry observed in the present process. For these tests a 400 kv electron-beam energy source was used to generate e_{aq}⁻ in mixtures of water and methane and the reaction products were monitored by GC.

A series of small-scale (200 to 300 ml) batch studies were conducted to examine the conversion of water and methane to hydrogen and higher molecular weight hydrocarbons upon exposure to radiation from a low energy electron-beam. The electron beam studies were conducted using a 400 KeV, 900 Watt accelerator.

In these tests, the effects of electron-beam radiation on liquid- and vapor-phase water, methane, and a mixture of water and methane were investigated. Seven 300-ml polyvinylfluoride Teldar gas-sampling bags were filled with water, nitrogen or methane, and each bag exposed to multiple short exposures of 300 kv electron-beam radiation. Some samples were heated with a hot air gun prior to irradiation and placed on a hand warmer to increase the water vapor content of the gas mixture during exposure to the electron beam. After each exposure, a gas sample was collected and analyzed by a portable GC for H₂, O₂, N₂, CO, CO₂, CH₄, C₂H₂, C₂H₄, C₂H₆, and C₃H₈ content. The contents of the Tedlar bags were analyzed by GC/MS for higher molecule weight hydrocarbons (greater than 4 carbons). A summary of samples used in the electron-beam studies is provided in the following table.

TABLE 2
Batch Electron-Beam Test Samples

| Study | | |
|---|---|---|
| No. | Components | Purpose |
| 1 | N₂ | Test Control, Ambient Temperature and Heated |
| 2 | H₂O/N₂ | H₂O Conversion to H₂, Ambient Temp |
| 3 | H₂O/N₂ | H₂O Conversion to H₂, Heated |
| 4 | CH₄ | CH₄ Conversion to H₂ and Hydrocarbons, Ambient |
| | | Temp |
| 4a | CH₄ | CH₄ Conversion to H₂ and Hydrocarbons, Ambient |
| | | Temp |
| 5 | CH₄ | CH₄ Conversion to H₂ and Hydrocarbons, Heated |
| 6 | H₂O/CH₄ | H₂O/CH₄ Conversion to H₂ and Hydrocarbons, |
| | | Ambient Temp |
| 7 | H₂O/CH₄ | H₂O/CH₄ Conversion to H₂ and Hydrocarbons, |
| | | Heated |
| 7a | H₂O/CH₄ | Long Exposure H₂O/CH₄ Conversion to H₂ and |
| | | Hydrocarbons, Heated |

A summary of results from the electron-beam studies is provided in Table 3 and each of the individual studies is described in more detail below. Increased hydrogen production was achieved when heated mixtures of deionized water and methane were irradiated at elevated beam current. Hydrogen production rates of as high as 300 moles of molecular hydrogen per mole of electron radiation were attained when heated water/methane mixtures were exposed to a 0.100 milliamp, 300 kv beam. These conditions produce hydrogen, propane and highly-branched saturated hydrocarbons; similar to the suite of products produced during the photochemical studies.

TABLE 3
Electron Beam Test Results

| | Test Number | | | | | | | | |
|---|---|---|---|---|---|---|---|---|---|
| | 1 | 2 | 3 | 4 | 4a | 5 | 6 | 7 | 7a |
| Matrix | Nitrogen | Nitrogen | Nitrogen | Methane | Methane | Methane | Methane | Methane | Methane |
| | | Water | Water | Water | Water | Water | | | |
| Heat | No | No | Yes | No | No | Yes | No | Yes-Heating | Yes-Heating |
| | | | | | | | | Pad | Pad |
| Exposure No.1 | | | | | | | | | |
| Current, mA | 0.05 | 0.025 | 0.025 | 0.025 | 0.1 | 0.025 | 0.025 | 0.1 | 0.025 |
| Dosage, | 2.7 | 0.8 | 0.8 | 0.8 | 5.0 | 0.8 | 0.8 | 5.0 | 0.8 |
| Megarad/min | | | | | | | | | |
| Time, min | 1.0 | 1.0 | 1.0 | 1.0 | 3.0 | 1.0 | 1.0 | 3.0 | 1.0 |
| Magrad for | 2.7 | 0.8 | 0.8 | 0.8 | 15.0 | 0.8 | 0.8 | 15.0 | 0.8 |
| Exposure | | | | | | | | | |
| Total Megarad | 2.7 | 0.8 | 0.8 | 0.8 | 15.0 | 0.8 | 0.8 | 15.0 | 0.8 |
| Hydrogen | 0.023% | 0.010% | 0.010% | 0.020% | 0.31% | 0.015% | 0.019% | 0.60% | 0.018% |
| H2/e- | 67 | 59 | 59 | 113 | 149 | 88 | 111 | 288 | 102 |
| Exposure No.2 | | | | | | | | | |
| Current, mA | 0.1 | 0.025 | 0.025 | 0.025 | 0.1 | 0.025 | 0.025 | 0.1 | 0.025 |
| Dosage, | 5.0 | 0.8 | 0.8 | 0.8 | 5.0 | 0.8 | 0.8 | 5.0 | 0.8 |
| Megarad/min | | | | | | | | | |
| Time, min | 10.0 | 2.0 | 2.0 | 2.0 | 7.0 | 2.0 | 2.0 | 7.0 | 2.0 |
| Magrad for | 50.0 | 1.6 | 1.6 | 1.6 | 35.0 | 1.6 | 1.6 | 35.0 | 1.6 |
| Exposure | | | | | | | | | |
| Total Megarad | 52.7 | 2.4 | 2.4 | 2.4 | 50.0 | 2.4 | 2.4 | 50.0 | 2.4 |
| Hydrogen | 0.20% | 0.027% | 0.035% | 0.055% | 1.07% | 0.047% | 0.056% | 1.82% | 0.067% |
| H2/e- | | | | | | | | | |
| For Exposure | 25 | 48 | 72 | 100 | 155 | 89 | 106 | 251 | 117 |
| Total | 27 | 52 | 68 | 104 | 153 | 89 | 108 | 262 | 113 |
| Matrix | Nitrogen | Nitrogen | Nitrogen | Methane | Methane | Methane | Methane | Methane | Methane |
| | | Water | Water | Water | Water | Water | | | |
| Heat | No | No | Yes | No | No | Yes | No | Yes-Heating | Yes-Heating |
| | | | | | | | | Pad | Pad |
| Exposure No.3 | | | | | | | | | |
| Current, mA | | | 0.1 | 0.05 | | | | | 0.05 |
| Dosage, | | | 5 | 2.7 | | | | | 2.7 |
| Megarad/min | | | | | | | | | |
| Time, min | | | 10 | 1.0 | | | | | 1.0 |
| Magrad for | | | 50 | 2.7 | | | | | 2.7 |
| Exposure | | | | | | | | | |
| Total Megarad | | | 52.4 | 5.1 | | | | | 5.1 |
| Hydrogen | | | 0.497% | 0.11% | | | | | 0.134% |
| H2/e- | | | | | | | | | |
| For Exposure | | | 66 | 142 | | | | | 216 |
| Total | | | 66 | 121 | | | | | 154 |
| Exposure No.4 | | | | | | | | | |
| Current, ma | | | | 0.05 | | | | | 0.05 |
| Dosage, | | | | 2.7 | | | | | 2.7 |
| Megarad/min | | | | | | | | | |
| Time, min | | | | 2.0 | | | | | 2.0 |
| Magrad for | | | | 5.4 | | | | | 5.4 |
| Exposure | | | | | | | | | |
| Total Megarad | | | | 10.5 | | | | | 10.5 |
| Hydrogen | | | | 0.19% | | | | | 0.26% |
| H2/e- | | | | | | | | | |
| For Exposure | | | | 132 | | | | | 182 |
| Matrix | Nitrogen | Nitrogen | Nitrogen | Methane | Methane | Methane | Methane | Methane | Methane |
| | | Water | Water | Water | Water | Water | | | |
| Heat | No | No | Yes | No | No | Yes | No | Yes-Heating | Yes-Heating |
| | | | | | | | | Pad | Pad |
| Total | | | | 128 | | | | | 166 |
| Exposure No.5 | | | | | | | | | |
| Current, ma | | | | 0.1 | | | | | 0.1 |
| Dosage, | | | | 5.0 | | | | | 5.0 |
| Megarad/min | | | | | | | | | |
| Time, min | | | | 1.0 | | | | | 1.0 |
| Magrad for | | | | 5.0 | | | | | 5.0 |
| Exposure | | | | | | | | | |
| Total Megarad | | | | 15.5 | | | | | 15.5 |
| Hydrogen | | | | 0.36% | | | | | 0.47% |
| H2/e- | | | | | | | | | |
| For Exposure | | | | 230 | | | | | 302 |
| Total | | | | 159 | | | | | 210 |
| Exposure No.6 | | | | | | | | | |
| Current, ma | | | | 0.1 | | | | | 0.1 |
| Dosage, | | | | 5.0 | | | | | 5.0 |
| Megarad/min | | | | | | | | | |
| Time, min | | | | 2.0 | | | | | 2.0 |
| Magrad for | | | | 10.0 | | | | | 10.0 |
| Exposure | | | | | | | | | |
| Total Megarad | | | | 25.5 | | | | | 25.5 |
| Hydrogen | | | | 0.61% | | | | | 0.88% |
| H2/e- | | | | | | | | | |
| Matrix | Nitrogen | Nitrogen | Nitrogen | Methane | Methane | Methane | Methane | Methane | Methane |
| | | Water | Water | Water | Water | Water | | | |
| Heat | No | No | Yes | No | No | Yes | No | Yes-Heating | Yes-Heating |
| | | | | | | | | Pad | Pad |
| For Exposure | | | | 178 | | | | | 294 |
| Total | | | | 166 | | | | | 243 |
| Exposure No.7 | | | | | | | | | |
| Current, ma | | | | | | | | | 0.1 |
| Dosage, | | | | | | | | | 5 |
| Megarad/min | | | | | | | | | |
| Time, min | | | | | | | | | 30 |
| Magrad for | | | | | | | | | 150 |
| Exposure | | | | | | | | | |
| Total Megarad | | | | | | | | | 175.5 |
| Hydrogen | | | | | | | | | 4.95% |
| H2/e- | | | | | | | | | |
| For Exposure | | | | | | | | | 193 |
| Total | | | | | | | | | 235 |

Study No. 1: Control Sample.

The results from Study No. 1 were used to establish control data and investigate deleterious effects the electron-beam radiation may have on the integrity of the sample bag. For this test, a 300 ml Tedlar gas sampling bag containing approximately 200 ml N₂ was irradiated at 0.05 milliamp for one minute and 0.1 milliamp for ten minutes. The sampling bag developed a light golden-brown discoloration which darkened after each exposure. Analysis of the treated gas identified nitrogen as the only major constituent and minor amounts of hydrogen (0.20%), acetaldehyde, 1,3-Dioxolane, benzene, and heptane. The hydrogen and trace hydrocarbons were most likely decomposition products of the polyvinylfluoride Tedlar sample bag.

Study No 2: Water at Ambient Temperature.

In Study No. 2 the effects of exposure period and radiation dosage on hydrogen production from ambient temperature water was investigated. For this test, the sampling bag was charged with 20 ml of deionized water and approximately 200 ml of N₂ and irradiated for 3 minutes at 300 kv and 0.025 milliamps. The irradiated sampling bag developed a light-brown discoloration and contained 0.027% hydrogen.

Study No. 3: Increased Water Vapor.

The effect of increased temperature and current on electron-beam induced hydrogen conversion of water was investigated in Study No. 3. For this test, a gas sampling bag containing 20 ml of deionized water and approximately 200 ml of N₂ was heated with a hot air gun and irradiated for 1- and 2-minute exposures at a current of 0.025 milliamp and one 5-minute exposure at 0.100 milliamp. Results from this study showed increased temperature or beam current did not significantly effect hydrogen production rate.

Study No. 4 and 5. Methane Conversion at Ambient and Higher Temperatures.

The effects of electron-beam radiation on methane at ambient and higher temperatures were examined in Studies No. 4, No. 4a and No. 5. For these tests, gas sampling bags containing approximately 200 ml of ultra high purity methane (99.97%) were irradiated by an electron beam at various currents and exposures periods. During Study No. 5, the Tedlar bag was heated with a hot air gun prior to each exposure.

Results from these tests indicated methane is converted to highly-branched alkanes and hydrogen when exposed to 300 kv electron beam radiation. Increased conversion rates are achieved at higher beam currents. Elevated temperatures had no significant effect on the methane conversion rates. Study No. 6 and 7. Methane and Water at Ambient and higher Temperatures. During Study No. 6 and No. 7 and No. 7a, sampling bags containing approximately 20 ml deionized water and 200 ml methane were exposed to 300 kv electron-beam radiation at ambient (Study No. 6) and higher temperatures (Study No. 7a) and increased current (Study No. 7). These tests demonstrated that increased methane conversion rates are achieved when a mixture of water and methane is irradiated at elevated temperature and increased beam current.

In Study 7a, a heated mixture of methane and water was irradiated at various currents for an extended period (39-minute total exposure). The treated gas contained methane, 5% hydrogen, ethane, propane, and highly-branched saturated hydrocarbons in the C₄ to C₁₀ molecular weight range. No unsaturated hydrocarbons, oxygenated hydrocarbons, or aromatic compounds were identified as reaction products.

### Example 3

### Gamma Radiation Gas-To-Liquid Studies

A series of batch irradiation tests was conducted to examine the effects of gamma radiation on ultra-pure methane (99.97%) and methane/water mixtures. These tests were conducted to demonstrate that gamma radiation can be used to generate e_{aq}⁻ initiation sites and examine the effects of free radical scavengers on the active sites. In these studies, methane was used to scavenge free radicals from active e_{aq}⁻ sites to produce reactive products, molecular hydrogen and higher molecular weight hydrocarbons. These tests were conducted with a ⁶⁰Co gamma radiation source at a dose rate of 20 kRad/min. Four samples of Ultra High Purity methane (99.97%) and methane/water mixtures were prepared in ¾-inch diameter 6-inch long stainless steel vials. The vials were placed in a gamma irradiator for 16 hours and the treated gases analyzed by GC and GC/MS.

A description of the samples and hydrogen levels in the treated gases is provided in the following table.

TABLE 4
Description of Gas Samples and Hydrogen Levels
in the Treated Gases

| | Deionized | Methane Pressure, psig | | Hydrogen in |
|---|---|---|---|---|
| Sample No. | Water | Initial | Final | Treated Gas, % |
| 1 | None | 25 | 20 | 0.078 |
| 2 | 10 | 25 | 21 | 1.33 |
| 3 | 10 | 50 | 47 | 1.67 |
| 4 | 10 | 80 | 76 | 2.64 |

These results indicate that hydrogen is generated when ultra-pure methane is exposed to gamma radiation. Hydrogen generation is increased by over 17 fold when water is added to the purified methane. Increased hydrogen production was also observed at higher gas pressures.

GC/MS analysis identified propane, 2,2-dimethyl propane and 2,2-dimethyl butane as the primary hydrocarbon products of irradiated, 25 psig methane gas (Sample No. 1). When water was added to 25 psig methane, higher molecular weight branched hydrocarbons in the C7 to C9 range are produced. Increased amounts of higher molecular weight hydrocarbon products are produced as the methane pressure is increased to 50 and 80 psig. The distribution of hydrocarbon products is summarized in the following table.

TABLE 5
Distribution of Major Condensed Hydrocarbon Products

| | | Major Condensed Hydrocarbons, % | | | |
|---|---|---|---|---|---|
| | | No. 1 | No. 2 | No. 3 | No. 4 |
| | Molecular | 25 psig | 25 psig | 50 psig | 80 psig |
| Constituent | Weight | No H₂O | H₂O | H₂O | H₂O |
| Propane | 44 | 74% | 69% | 25% | 20% |
| 2-Methylpropane | 58 | | | | |
| Neopentane | 72 | | | | |
| 2-Methylbutane | 72 | | | | |
| 2,2-Dimethylbutane | 86 | | | | |
| 2,4- | 100 | | 2% | | |
| Dimethylpentane | | | | | |
| 3,3- | 100 | | 2% | | |
| Dimethylpentane | | | | | |
| 2,2,3,3- | 114 | | 5% | 4% | 5% |
| 2,2,3,3,- | 128 | | 7% | 10% | 9% |
| Branched C9s and | 128 to 142 | | | 33.4% | |
| C10s | | | | | |
| Branched C9s and | 128 to 198 | | | | 38% |
| C14s | | | | | |

### Example 4

### Nonthermal Plasma

Continuous-flow studies were conducted to examine methane chemistry and e_{aq}⁻ generation in a non-thermal plasma. A non-thermal discharge is characterized by a large number of free electrons that are accelerated through a low temperature gas by a large electrical potential (5 to 20 Kv). A commercial corona-discharge ozone generator (Ozomax OZO 4 LT) was employed to generate the non-thermal discharge used in this study. This generator was fitted with two dielectric barrier discharge (DBD) electrodes designed to produce 20 gram-per-hour ozone at an oxygen feed rate of 3.5 liters-per-minute and 200 watts power.

In the initial plasma study, ultra-pure methane was processed through the plasma generator at a pressure of 3 psig, flow rate of 325 ml/min, and 1.5 amps at an input voltage of 79 volts. After 20 minutes the product gas stream was sampled and analyzed for hydrogen and hydrocarbon content. The product gas contained 1.95 mole % hydrogen. Ethane, propane, and normal and branched hydrocarbons in the C4 to C10 range were also identified in the product gas stream. These products are similar to the constituents from the photochemcial studies of Example 1.

In the second study, deionized/deoxygenated water was fed to the plasma generator at a flow rate of 0.6 ml/min at the same ultra-pure methane flow rate, pressure, and amperage as the initial non-thermal plasma test. The product gas from this test contained 3.97% hydrogen and a suite of hydrocarbon constituents identified in the initial non-thermal plasma study. No unsaturated hydrocarbons, oxygenated hydrocarbons, or aromatic compounds were identified as reaction products.

The principles, preferred embodiments and modes of operation of the present invention have been described in the foregoing specification. The invention which is intended to be protected herein should not, however, be construed as limited to the particular forms disclosed, as these are to be regarded as illustrative rather than restrictive. Variations and changes may be made by those skilled in the art without departing from the spirit of the present invention. Accordingly, the foregoing best mode of carrying out the invention should be considered exemplary in nature and not as limiting to the scope and spirit of the invention as set forth in the appended claims.

What is claimed is:

1. A process of converting methane to higher molecular weight hydrocarbons, comprising:
a. forming hydrogen and hydroxyl radicals; and,
b. contacting the hydrogen and hydroxyl radicals with methane, wherein the hydrogen and hydroxyl radicals react with the methane to form hydrogen and higher molecular weight hydrocarbon products.

2. The process of claim 1, wherein the methane is a gas and the higher molecular weight products are a liquid.

3. The process of claim 1, wherein the higher molecular weight hydrocarbon products comprise hydrocarbons having between 4 and 29 carbons.

4. The process of claim 1, wherein the higher molecular weight hydrocarbon products comprise hydrocarbons having between 9 and 14 carbons.

5. The process of claim 1, wherein the process is conducted in the absence of a molecular oxidant.

6. The process of claim 1, wherein the process is conducted in the presence of a molecular oxidant and wherein the higher molecular weight hydrocarbons are oxygenated.

7. The process of claim 1, wherein the process is conducted in a reducing atmosphere.

8. The process of claim 1, wherein the hydrogen and hydroxyl radicals are formed by contacting hydrated electrons with water.

9. The process of claim 8, wherein the hydrated electrons are present in a spur comprising hydrated electrons (e_{aq}⁻), H and OH.

10. The process of claim 8, wherein the hydrated electrons are formed by contacting a mixture of methane and water with an energy source.

11. The process of claim 10, wherein the water is present as water vapor.

12. The process of claim 10, wherein the water vapor contains methane.

13. The process of claim 10, wherein the water has a temperature in the range of about 15° C. to about 50° C.

14. The process of claim 10, wherein the water has a temperature in the range of about 50° C. to about 150° C.

15. The process of claim 10, wherein the water has a temperature of greater than about 150° C.

16. The process of claim 10, wherein the methane and water mixture is maintained at atmospheric pressure.

17. The process of claim 10, wherein the methane and water mixture is maintained at a pressure less than atmospheric pressure.

18. The process of claim 10, wherein the methane and water mixture is maintained at a pressure greater than atmospheric pressure.

19. The process of claim 18, wherein the methane and water mixture is maintained at about 80 psi.

20. The process of claim 10, wherein the methane and water are present in a ratio of between about 1:5 to about 5:1.

21. The process of claim 10, wherein the methane and water are present in a ratio of about 1:1.

22. The process of claim 10, wherein the contacting step comprises exposing the methane and water mixture to gamma radiation.

23. The process of claim 22, wherein the gamma radiation is at a dose rate of between about 10 kRad/min and about 50 kRad/min.

24. The process of claim 10, wherein the contacting step comprises exposing the methane and water mixture to ultra violet radiation.

25. The process of claim 24, wherein the exposure to ultra violet radiation results in dissociation of water.

26. The process of claim 24, wherein the ultra violet radiation has a wavelength shorter than about 380 nm.

27. The process of claim 24, wherein the ultra violet radiation has a wavelength range between about 150 nm and about 280 nm.

28. The process of claim 24, wherein said step of exposing is conducted in the presence of a photocatalyst.

29. The process of claim 10, wherein the contacting step comprises exposing the methane and water mixture to an electron-beam.

30. The process of claim 28, wherein the electron beam is an at least about 300 kv electron-beam.

31. The process of claim 10, wherein the contacting step comprises exposing the methane and water mixture to dielectric barrier plasma discharge.

32. The process of claim 10, wherein the contacting step comprises exposing the methane and water mixture to an electrical discharge.

33. The process of claim 31, wherein the electrical discharge is a corona electrical discharge.

34. The process of claim 1, wherein the process of forming hydrogen and hydroxyl radicals comprises sonication.

35. A process of converting high molecular weight hydrocarbons to lower molecular weight hydrocarbons comprising:
a. forming hydrogen and hydroxyl radicals; and,
b. contacting the hydrogen and hydroxyl radicals with high molecular weight hydrocarbons, wherein the hydrogen and hydroxyl radicals react with the high molecular weight hydrocarbons to form hydrogen and lower molecular weight hydrocarbon products.

36. The process of claim 35, wherein the high molecular weight hydrocarbons comprise hydrocarbons having greater than 30 carbons.

37. The process of claim 35, wherein the lower molecular weight hydrocarbon products comprise hydrocarbons having between 4 and 29 carbons.

38. The process of claim 35, wherein the lower molecular weight products comprise hydrocarbons having between 9 and 14 carbons.

39. The process of claim 35, wherein the hydrogen and hydroxyl radicals are formed by contacting hydrated electrons with water.

40. The process of claim 35, wherein hydrated electrons are formed by contacting a mixture of high molecular weight hydrocarbons and water with an energy source.

41. The process of claim 40, wherein the water is present as water vapor.

42. The process of claim 41, wherein the water vapor contains high molecular weight hydrocarbons.

43. The process of claim 40, wherein the water has a temperature in the range of about 15° C. to about 50° C.

44. The process of claim 40, wherein the water has a temperature in the range of about 50° C. to about 250° C.

45. The process of claim 40, wherein the water has a temperature of greater than about 250° C.

46. The process of claim 40, wherein the high molecular weight hydrocarbons and water mixture is maintained at atmospheric pressure.

47. The process of claim 40, wherein the high molecular weight hydrocarbons and water mixture is maintained at a pressure greater than atmospheric pressure.

48. The process of claim 40 wherein the high molecular weight hydrocarbons and water mixture is maintained at about 80 psi.

49. The process of claim 40, wherein the high molecular weight hydrocarbons and water mixture is maintained at a pressure less than atmospheric pressure.

50. The process of claim 40, wherein the high molecular weight hydrocarbons and water are present in a ratio of about 1:1.

51. The process of claim 40, wherein the energy source is gamma radiation.

52. The process of claim 40, wherein the energy source is an electron beam.

53. The process of claim 40, wherein the energy source is ultra violet radiation.

54. The process of claim 53, wherein the ultra violet radiation has a wavelength shorter than about 380 nm.

55. The process of claim 53, wherein the ultra violet radiation has a wavelength range between about 150 nm and about 280 nm.

56. The process of claim 53, wherein said step of contacting is conducted in the presence of a photocatalyst.

57. The process of claim 40, wherein the energy source is a dielectric barrier discharge.

58. The process of claim 40, wherein the energy source is an electrical discharge.

59. The process of claim 40, wherein the energy source is a corona electrical discharge.

60. The process of claim 35, wherein the process is conducted in the absence of a molecular oxidant.

61. The process of claim 35, wherein the process is conducted in a reducing atmosphere.

62. A process of converting low molecular weight hydrocarbons to higher molecular weight hydrocarbons, comprising:
a. forming hydrated electrons; and,
b. contacting the hydrated electrons with a starting material comprising low molecular weight hydrocarbons, wherein the hydrated electrons react with the low molecular weight hydrocarbons to form hydrogen and higher molecular weight hydrocarbon products.

63. The process of claim 62, wherein the low molecular weight hydrocarbons are selected from the group consisting of methane, ethane, propane, butane and mixtures thereof.

64. The process of claim 62, wherein the starting material is natural gas.
